(19) United States
(12) Patent Application Publication
(10) Pub. No.: US 2025/0378282 A1
(43) Pub. Date: Dec. 11, 2025

(54) LARGE LANGUAGE MODEL-BASED MEDICAL EXAMINATION CONCLUSION GENERATION METHOD AND APPARATUS

(71) Applicant: Alipay (Hangzhou) Information Technology Co.., Ltd., Hangzhou (CN)

(72) Inventor: Yujiao LI, Hangzhou (CN)

(21) Appl. No.: 19/227,847

(22) Filed: Jun. 4, 2025

(30) Foreign Application Priority Data
Jun. 5, 2024 (CN) 202410725950.4

(51) Int. Cl. G06F 40/40; A61B 5/00; A61B 5/055; G16H 10/60; G16H 15/00; G16H 70/20

(52) U.S. Cl. CPC G06F 40/40; A61B 5/055; A61B 5/4381; G16H 10/60; G16H 15/00; G16H 70/20

(57) ABSTRACT

A large language model-based medical examination conclusion generation method includes: obtaining a target manifestation text corresponding to a target medical examination; extracting medical examination inference knowledge that matches the target manifestation text from a medical examination inference knowledge base, where the medical examination inference knowledge includes a manifestation text and a conclusion text corresponding to a medical examination; constructing a sample based on the extracted medical examination inference knowledge, and constructing a prompt text based on the sample and the target manifestation text; and inputting the prompt text into a large language model, and outputting, by using the large language model, a target conclusion text that corresponds to the target medical examination and that is obtained by performing inference based on the target manifestation text and under guidance of the sample.

### CROSS-REFERENCE TO RELATED APPLICATIONS

This application claims priority to Chinese Patent Application No. 202410725950.4, filed on Jun. 5, 2024, the entire content of which is incorporated herein by reference.

### TECHNICAL FIELD

The present disclosure relates to the field of artificial intelligence technologies, and in particular, to a large language model-based medical examination conclusion generation method and apparatus.

### BACKGROUND

In actual medical scenarios, medical examinations refer to a series of procedures and tests conducted by doctors or medical professionals on patients to evaluate health conditions, diagnose diseases, monitor disease progression, or determine treatment effectiveness. There can be a plurality of types of medical examinations, for example, physical examinations (observation of an appearance of an examinee, auscultation, and measurement of vital signs), laboratory tests (a blood test, a urine test, and a stool test), imaging examinations (X-ray, computed tomography, magnetic resonance imaging, and ultrasound), functional tests (electrocardiogram, pulmonary function test, and endoscopy), and special examinations (positron emission tomography and a bone density examination).

After a medical examination is conducted on a patient, a medical report of the conducted medical examination is usually provided. Content of the medical report can include not only personal information (for example, a name, contact information, and a medical record number) of the examinee, but also a medical examination manifestation and a medical examination conclusion. The medical examination manifestation is a specific condition or phenomenon directly observed in a medical examination process by using various examination means (for example, a laboratory test, an imaging examination, and a functional test). The manifestation can be a specific value (for example, a certain protein content in blood), a form description (for example, a shape and distribution of a pulmonary shadow on an X-ray film), a function evaluation result (for example, a waveform change in an electrocardiogram), or another measurable indicator. The medical examination conclusion is a summary judgment of a health condition or a disease status of the examinee obtained after comprehensive analysis is performed based on the medical examination manifestation and other clinical information, and usually clearly indicates whether examination findings suggest the presence of a disease, the severity of a condition, treatment effectiveness evaluation, or another medical judgment. In short, the medical examination conclusion is a high-level summary of the medical examination manifestation, and is used to provide a clear basis for diagnosis or exclusion of a specific disease for a doctor and a patient. Therefore, in actual application, how to efficiently and accurately obtain a medical examination conclusion corresponding to a medical examination manifestation becomes an urgent problem to be resolved.

### SUMMARY

According to a first aspect of the present disclosure, there is provided a large language model-based medical examination conclusion generation method. The method includes: obtaining a target manifestation text corresponding to a target medical examination; extracting medical examination inference knowledge that matches the target manifestation text from a medical examination inference knowledge base, where the medical examination inference knowledge includes a manifestation text and a conclusion text corresponding to a medical examination; constructing a sample based on the extracted medical examination inference knowledge, and constructing a prompt text based on the sample and the target manifestation text; and inputting the prompt text into a large language model, and outputting, by using the large language model, a target conclusion text that corresponds to the target medical examination and that is obtained by performing inference based on the target manifestation text and under guidance of the sample.

According to a second aspect of the present disclosure, there is provided a large language model-based medical examination conclusion generation apparatus. The apparatus includes: a processor; and a memory storing instructions executable by the processor. The processor is configured to: obtain a target manifestation text corresponding to a target medical examination; extract medical examination inference knowledge that matches the target manifestation text from a medical examination inference knowledge base, where the medical examination inference knowledge includes a manifestation text and a conclusion text corresponding to a medical examination; construct a sample based on the extracted medical examination inference knowledge, and construct a prompt text based on the sample and the target manifestation text; and input the prompt text into a large language model, and output, by using the large language model, a target conclusion text that corresponds to the target medical examination and that is obtained by performing inference based on the target manifestation text and under guidance of the sample.

According to a third aspect of the present disclosure, there is provided a non-transitory computer-readable storage medium. The computer-readable storage medium stores computer instructions that, when executed by a processor, cause the processor to perform the method according to the first aspect described above.

In the above-mentioned manner, no doctor or medical professional needs to manually determine a medical examination conclusion corresponding to a medical examination based on a medical examination manifestation corresponding to the medical examination, but the large language model can be used to perform inference based on the medical examination manifestation, to generate a corresponding medical examination conclusion, thereby improving efficiency of generating the medical examination conclusion. In addition, a sample can be generated based on medical examination inference knowledge that matches the medical examination manifestation, and the large language model can learn how to generate a corresponding conclusion text based on a manifestation text shown in the sample, and then perform inference based on the medical examination manifestation, to generate a corresponding medical examination conclusion, thereby improving pertinence of the generated medical examination conclusion and improving accuracy of the generated medical examination conclusion. Moreover, a prompt text of the large language model can be further constructed based on the constructed sample and the medical examination manifestation, that is, the sample can be provided to the large language model through few-shot learning, so that the language model can effectively learn on a limited and small quantity of samples, thereby reducing difficulty of training the large language model.

### BRIEF DESCRIPTION OF DRAWINGS

The following briefly describes the accompanying drawings of the present disclosure.

FIG. 1 is a schematic diagram of a large language model-based medical examination conclusion generation procedure according to an example embodiment.

FIG. 2 is a flowchart of a large language model-based medical examination conclusion generation method according to an example embodiment.

FIG. 3 is a schematic diagram of an inference step of inferring a conclusion text from a manifestation text according to an example embodiment.

FIG. 4 is a block diagram of an electronic device according to an example embodiment.

FIG. 5 is a block diagram of a large language model-based medical examination conclusion generation apparatus according to an example embodiment.

### DESCRIPTION OF EMBODIMENTS

Example embodiments are described in detail herein and shown in the accompanying drawings. When the following description relates to the accompanying drawings, unless specified otherwise, the same numbers in different accompanying drawings represent the same or similar elements. Implementations described in the following example embodiments do not represent all implementations consistent with the present disclosure. On the contrary, the implementations are merely examples consistent with some aspects of one or more embodiments of the present disclosure.

It should be noted that in other embodiments, steps of a corresponding method are not necessarily performed based on a sequence shown and described in this disclosure. In some other embodiments, the method can include more or fewer steps than those described in this disclosure. In addition, a single step described in this disclosure may be split into a plurality of steps in other embodiments for description; and a plurality of steps described in this disclosure may be combined into a single step in other embodiments for description.

In actual medical scenarios, medical examinations refer to a series of procedures and tests conducted by doctors or medical professionals on patients to evaluate health conditions, diagnose diseases, monitor disease progression, or determine treatment effectiveness. There can be a plurality of types of medical examinations, for example, physical examinations, laboratory tests, imaging examinations, functional tests, and special examinations.

A medical examination manifestation is a specific condition or phenomenon directly observed in a medical examination process by using various examination means. The manifestation can be a specific value, a form description, a function evaluation result, or another measurable indicator. A medical examination conclusion is a summary judgment of a health condition or a disease status of an examinee obtained after comprehensive analysis is performed based on the medical examination manifestation and other clinical information, and usually clearly indicates whether examination findings suggest the presence of a disease, the severity of a condition, treatment effectiveness evaluation, or another medical judgment. In short, the medical examination conclusion is a high-level summary of the medical examination manifestation, and is used to provide a clear basis for diagnosis or exclusion of a specific disease for a doctor and a patient.

The medical examination manifestation can be presented in various forms such as a text and an image (for example, an X-ray film and an electrocardiogram), and the medical examination conclusion is usually presented in a text form. The imaging examination is used as an example. In this case, the medical examination manifestation can include not only a radiographic image such as an X-ray film, a CT image, or an MRI image obtained by performing radiological scanning on an examinee, but also a descriptive text of a body part, an organ, or a lesion displayed in the obtained radiographic image. For example, if a magnetic resonance imaging examination is conducted on the prostate of the examinee, a medical examination manifestation corresponding to this examination can include not only an MRI image of the prostate of the examinee, but also the following descriptive text: The prostate is increased in size and protrudes upward toward the base of the bladder, no significant abnormal signal is observed in the prostate on T1WI, a nodular iso-to-slightly hyperintense signal is observed in the transition zone on the left side of the prostate on T2WI, DWI shows a high signal, and there is marked heterogeneous enhancement post-contrast administration. The medical examination conclusion can include a text describing a summary judgment of a health condition or a disease status of the examinee. For example, a medical examination conclusion corresponding to the magnetic resonance imaging examination on the prostate of the examinee can include the following text: Prostate hyperplasia is noted, PI-RADS classification is considered, prostate cancer is suspected with reference to clinical data, and further evaluation is needed.

For the medical examination conclusion, a doctor or a medical professional usually needs to manually view the medical examination manifestation, interpret and analyze the medical examination manifestation, and if necessary, refer to other clinical information to obtain the summary judgment of the health condition or the disease status of the examinee as the medical examination conclusion corresponding to the medical examination manifestation. This process includes understanding an initial manifestation from the medical examination manifestation and extracting the exact medical examination conclusion. This involves highly specialized and detailed work, and usually requires a significant amount of time and effort from the doctor or the medical professional.

Embodiments of this disclosure provide a technical solution for generating a medical examination conclusion based on a large language model (LLM), to efficiently and accurately obtain a medical examination conclusion corresponding to a medical examination manifestation, reduce burden on a doctor or a medical professional, and lower labor costs. For example, according to the technical solutions provided in this disclosure, a medical examination conclusion corresponding to a medical examination manifestation is generated by using a large language model based on an idea of few-shot learning.

The large language model is a deep learning model trained by using a large amount of text data, and can be used to generate a natural language text or understand a meaning of a natural language text. The large language model can process a plurality of natural language tasks, for example, text classification, named entity recognition, question answering, and dialogues, and is an important approach to artificial intelligence.

In the field of natural language processing, a large-scale text data set is usually referred to as a corpus. The corpus can include various types of text data, for example, literary works, academic papers, legal documents, news reports, daily dialogues, emails, and web forum posts. By learning from the text data in the corpus, the large language model can obtain and understand a rule and a pattern of a natural language, thereby implementing effective processing and generation of a human language.

The large language model usually uses a transformer architecture, that is, the large language model is usually a deep learning model based on the transformer architecture. The deep learning model based on the transformer architecture is a class of neural network models using the transformer architecture. Such a model performs excellently in fields such as natural language processing.

A transformer is a neural network model for sequence-to-sequence modeling. The transformer does not need to depend on a recursive structure, and can parallelize training and inference, accelerating a model processing speed. In the deep learning model based on the transformer architecture, a multi-layer transformer encoder is usually used to extract features from an input sequence, and a transformer decoder is used to convert the extracted features into an output sequence. In addition, in such a model, a self-attention mechanism is usually used to capture a long-range dependency in the input sequence, and a residual connection and a regularization method are used to accelerate training and improve model performance.

A pre-trained model is a large language model pre-trained on large-scale unlabeled text data. The pre-trained model is a general model and is not designed and optimized for a specific task. To enable the pre-trained model to adapt to a specific application scenario and task requirement, fine-tuning needs to be performed to improve performance of the model in a specific task. A large language model that is finally put into use is usually a model obtained by performing further fine-tuning based on the pre-trained model and performing supervised learning based on labeled text data. Pre-training and fine-tuning are complementary processes. Pre-training enables the model to have an extensive language understanding capability, while fine-tuning makes the model more professional and accurate in a specific task.

That is, a training process of the large language model can be divided into two phases: pre-training and fine-tuning. In the pre-training phase, pre-training can be performed on a large-scale unlabeled text data set (for example, network encyclopedia, network articles, and books) through unsupervised learning (for example, self-supervised learning). For example, a missing part or a next word can be predicted based on context, a statistical rule and a language structure such as semantics and syntax can be learned, and backpropagation and optimization algorithms (for example, a gradient descent method) can be used to minimize a prediction loss and iteratively update a model parameter, to gradually improve a language understanding capability of the model. In the fine-tuning phase, a corresponding supervised learning task (for example, text classification, named entity recognition, a question-answering system, or a dialogue system) can be selected based on a specific application scenario and a task requirement, and a task-specific text data set is prepared. Therefore, the pre-trained model can be used as a start point for fine-tuning, and fine-tuning training can be performed on the task-specific text data set through supervised learning. For example, the task can be executed based on the text data set, and the backpropagation and optimization algorithms (for example, the gradient descent method) can be used to minimize a loss used to measure performance of the model in processing a specific task and iteratively update the model parameter, to gradually improve the performance of the model in the specific task.

It should be noted that the pre-trained large language model is usually referred to as a foundation model of the large language model, and the fine-tuned large language model is referred to as a service model of the large language model. The language understanding capability learned by the large language model in the pre-training phase and the fine-tuning phase enables the large language model to understand, analyze, and combine text information to perform logical inference or knowledge inference, or resolve problems when facing complex problems or tasks. Such a capability is usually referred to as an inference capability of the large language model.

The large language model usually executes a specific task under guidance of a prompt text (which can be referred to as a prompt). The prompt text is an initial text or a text segment provided to the large language model to stimulate the model to generate a corresponding output. The prompt text can be used to clearly notify the large language model of a task that the large language model is expected to execute, for example, answering a question, simulating a dialogue, writing an article, or translating a text. In addition, the prompt text can provide necessary background information and context to the large language model, so that the large language model can understand logic, a style, a subject, or a position that should be followed when content is generated. Moreover, the prompt text can further stimulate the large language model to display its inherent knowledge reserve or specific language capability, for example, explaining complex concepts, citing regulations, or imitating a writing style of a specific writer.

Few-shot learning is an important branch in the field of machine learning and deep learning, and focuses on how to enable algorithms to learn effectively and generalize to unseen data with only a small quantity of training samples. In a conventional machine learning task, a model often requires a large amount of labeled data to achieve relatively good performance, and few-shot learning is intended to reduce this dependence on large-scale data. A key challenge for few-shot learning is how to capture inherent patterns and features of data from limited examples, to implement recognition of new categories or execution of tasks. Few-shot learning is widely applied to a plurality of fields such as image classification, object recognition, and natural language processing, and is especially applicable to scenarios in which a large amount of labeled data is difficult to obtain.

According to the technical solutions provided in this disclosure, for a target manifestation text corresponding to a target medical examination, medical examination inference knowledge that matches the target manifestation text can be first extracted from a medical examination inference knowledge base, where the medical examination inference knowledge can include a manifestation text and a conclusion text corresponding to a medical examination; then, a sample can be constructed based on the extracted medical examination inference knowledge, and a prompt text can be further constructed based on the sample and the target manifestation text; and finally, the prompt text can be input into a large language model, and a target conclusion text that corresponds to the target medical examination and that is obtained by performing inference based on the target manifestation text and under guidance of the sample can be output by using the large language model.

In the above-mentioned manner, no doctor or medical professional needs to manually determine a medical examination conclusion corresponding to a medical examination based on a medical examination manifestation corresponding to the medical examination, and the large language model can be used to perform inference based on the medical examination manifestation, to generate a corresponding medical examination conclusion, thereby improving efficiency of generating the medical examination conclusion. In addition, a sample can be generated based on medical examination inference knowledge that matches the medical examination manifestation, and the large language model can learn how to generate a corresponding conclusion text based on a manifestation text shown in the sample, and then perform inference based on the medical examination manifestation, to generate a corresponding medical examination conclusion, thereby improving pertinence of the generated medical examination conclusion and improving accuracy of the generated medical examination conclusion. Moreover, a prompt text of the large language model can be further constructed based on the constructed sample and the medical examination manifestation, that is, the sample can be provided to the large language model through few-shot learning, so that the language model can effectively learn on a limited and small quantity of samples, thereby reducing difficulty of training the large language model.

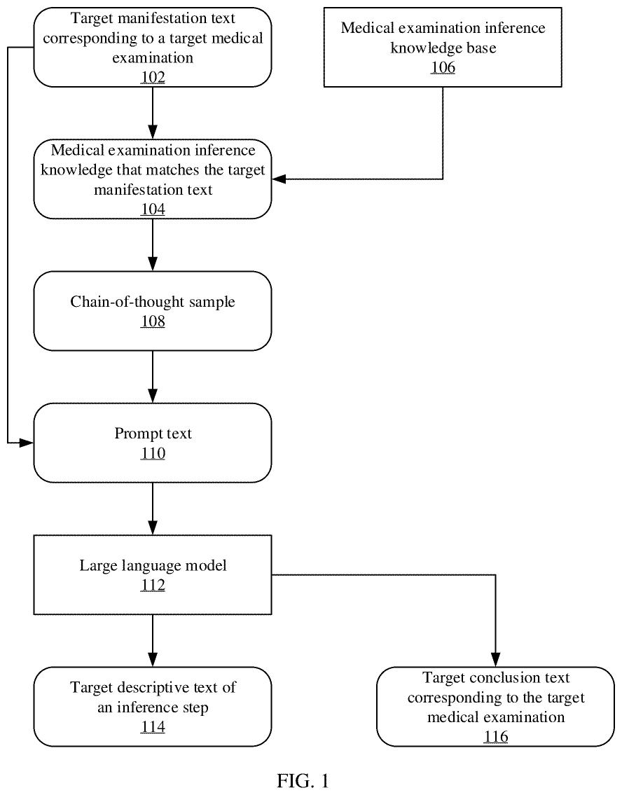

FIG. 1 is a schematic diagram of a large language model-based medical examination conclusion generation procedure according to an example embodiment.

In this embodiment, after one or more medical examinations (which can be referred to as target medical examinations) are conducted on an examinee, a medical examination manifestation corresponding to the target medical examination can be obtained. The medical examination manifestation can be presented in a text form, and is referred to as a target manifestation text 102. To obtain a medical examination conclusion corresponding to the medical examination manifestation as a medical examination conclusion corresponding to the target medical examination, the target manifestation text 102 can be obtained first, to obtain the medical examination conclusion by performing specific processing on the target manifestation text 102. The medical examination conclusion can also be presented in a text form, and is referred to as a target conclusion text.

When the target manifestation text 102 is obtained, to generate a sample that matches the target manifestation text 102 and enable a large language model to learn from the generated sample to obtain a more targeted inference capability, medical examination inference knowledge 104 that matches the target manifestation text 102 can be extracted from a medical examination inference knowledge base 106, to generate, based on the extracted medical examination inference knowledge 104, the sample that matches the target manifestation text 102. For example, the medical examination inference knowledge base 106 may be stored in a memory device.

The medical examination inference knowledge base 106 stores medical examination inference knowledge. For a medical examination inference knowledge entry, the medical examination inference knowledge entry can include a manifestation text (which can be specifically a text paragraph, a text chapter, etc.) and a conclusion text corresponding to a medical examination.

When the medical examination inference knowledge 104 that matches the target manifestation text 102 is extracted from the medical examination inference knowledge base 106, a sample 108 can be constructed based on the extracted medical examination inference knowledge 104, and a limited and small quantity of samples can be constructed. After the sample 108 is constructed, a prompt text 110 of the large language model can be further constructed based on the sample 108 and the target manifestation text 102.

In some embodiments, the constructed sample can be provided to the model through few-shot learning, that is, before the model receives an actual question, several complete examples including a question and an answer are first displayed, to direct the model to learn how to generate a corresponding answer based on a question.

When the prompt text 110 is constructed, the prompt text 110 can be input into the large language model, and a conclusion text, that is, the target conclusion text, that corresponds to the target medical examination and that is obtained by performing inference based on the target manifestation text included in the prompt text and under guidance of the sample included in the prompt text can be output by using the large language model. For example, the large language model can first learn from the sample to learn how to generate a corresponding conclusion text based on the manifestation text shown in the sample, and then perform inference based on the target manifestation text, to generate the target conclusion text.

For example, to constrain an inference process in which the large language model generates the conclusion text based on the manifestation text and enable formats of generated conclusion texts to be relatively consistent, for a medical examination inference knowledge entry stored in the medical examination inference knowledge base 106, the medical examination inference knowledge entry can include not only a manifestation text (which can be specifically a text paragraph, a text chapter, etc.) and a conclusion text corresponding to a medical examination, but also a descriptive text of an inference step of inferring the conclusion text from the manifestation text. In this case, a limited and small quantity of chain-of-thought samples 108 can be constructed based on the extracted medical examination inference knowledge 104. After the chain-of-thought sample 108 is constructed, the prompt text 110 of the large language model can be further constructed based on the chain-of-thought sample 108 and the target manifestation text 102.

In some embodiments, the chain-of-thought sample can direct the model to learn, by using a complete example of a question, an intermediate inference step, and an answer, to imitate such an inference pattern.

In this case, the large language model can output, in response to the prompt text, a descriptive text (which can be referred to as a target descriptive text) of an inference step of performing inference based on the target manifestation text and under guidance of the chain-of-thought sample, and simultaneously output the inferred target conclusion text. For example, the large language model can first learn from the chain-of-thought sample to learn an inference pattern shown in the chain-of-thought sample, and then imitate such an inference pattern, perform inference based on the target manifestation text, and output the target descriptive text used to describe the inference step in the inference process and the inferred target conclusion text.

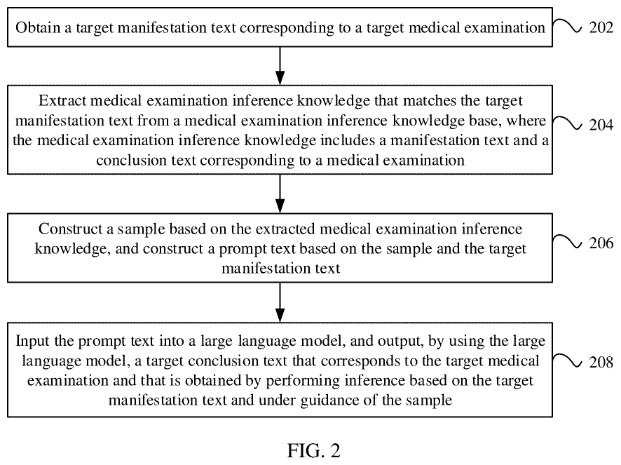

FIG. 2 is a flowchart of a large language model-based medical examination conclusion generation method according to an example embodiment.

In this embodiment, the large language model-based medical examination conclusion generation method can be applied to a server. The server can be a server that includes one independent physical host, or can be a server cluster that includes a plurality of independent physical hosts. Alternatively, the server can be a virtual server, a cloud server, etc. carried by a host cluster. Alternatively, the large language model-based medical examination conclusion generation method can be applied to an electronic device having a specific computing capability, for example, a tablet computer, a notebook computer, a desktop computer, a personal computer (PC), or a personal digital assistant (PDA).

As shown in FIG. 2, the large language model-based medical examination conclusion generation method can include the following steps.

Step 202: Obtain a target manifestation text corresponding to a target medical examination.

In this embodiment, after one or more medical examinations (which can be referred to as target medical examinations) are conducted on an examinee, a medical examination manifestation corresponding to the target medical examination can be obtained. The medical examination manifestation can be presented in a text form, and is referred to as a target manifestation text. To obtain a medical examination conclusion corresponding to the medical examination manifestation as a medical examination conclusion corresponding to the target medical examination, the target manifestation text can be obtained first, to obtain the medical examination conclusion by performing specific processing on the target manifestation text. The medical examination conclusion can also be presented in a text form, and is referred to as a target conclusion text.

Step 204: Extract medical examination inference knowledge that matches the target manifestation text from a medical examination inference knowledge base, where the medical examination inference knowledge includes a manifestation text and a conclusion text corresponding to a medical examination.

In this embodiment, when the target manifestation text is obtained, to generate a sample that matches the target manifestation text and enable a large language model to learn from the generated sample to obtain a more targeted inference capability, the medical examination inference knowledge that matches the target manifestation text can be extracted from the medical examination inference knowledge base, to generate, based on the extracted medical examination inference knowledge, the sample that matches the target manifestation text.

The medical examination inference knowledge base stores medical examination inference knowledge. For a medical examination inference knowledge entry, the medical examination inference knowledge entry can include a manifestation text (which can be specifically a text paragraph, a text chapter, etc.) and a conclusion text corresponding to a medical examination. For example, if a manifestation text corresponding to a medical examination is “The prostate is increased in size and protrudes upward toward the base of the bladder, no significant abnormal signal is observed in the prostate on T1WI, a nodular iso-to-slightly hyperintense signal is observed in the transition zone on the left side of the prostate on T2WI, DWI shows a high signal, and there is marked heterogeneous enhancement post-contrast administration”, a conclusion text corresponding to the medical examination can be “Prostate hyperplasia is noted, PI-RADS classification is considered, prostate cancer is suspected with reference to clinical data, and further evaluation is needed”.

In some embodiments, to constrain an inference process in which the large language model generates the conclusion text based on the manifestation text and enable formats of generated conclusion texts to be relatively consistent, for a medical examination inference knowledge entry stored in the medical examination inference knowledge base, the medical examination inference knowledge entry can include not only a manifestation text (which can be specifically a text paragraph, a text chapter, etc.) and a conclusion text corresponding to a medical examination, but also a descriptive text of an inference step of inferring the conclusion text from the manifestation text. For example, if a manifestation text corresponding to a medical examination is “The prostate is increased in size and protrudes upward toward the base of the bladder, no significant abnormal signal is observed in the prostate on T1WI, a nodular iso-to-slightly hyperintense signal is observed in the transition zone on the left side of the prostate on T2WI, DWI shows a high signal, and there is marked heterogeneous enhancement post-contrast administration”, a descriptive text of an inference step of performing inference based on the manifestation text can be “It can be learned from the prostate being increased in size and protruding upward toward the base of the bladder that the prostate is increased in size to a degree of protruding upward toward the base of the bladder, indicating prostatic hyperplasia; and it can be learned from no significant abnormal signal being observed in the prostate on T1WI, a nodular iso-to-slightly hyperintense signal being observed in the transition zone on the left side of the prostate on T2WI, DWI showing a high signal, and there being marked heterogeneous enhancement post-contrast administration that there is a nodular signal abnormality in the transition zone on the left side of the prostate, PI-RADS classification is considered, prostate cancer is suspected with reference to clinical data, and further evaluation is needed”. Correspondingly, a conclusion text corresponding to the medical examination can be “Prostate hyperplasia is noted, PI-RADS classification is considered, prostate cancer is suspected with reference to clinical data, and further evaluation is needed”.

In some embodiments, as described above, a medical examination inference knowledge entry stored in the medical examination inference knowledge base can include a manifestation text corresponding to a medical examination. In this case, when the medical examination inference knowledge that matches the target manifestation text is extracted from the medical examination inference knowledge base, a text similarity between the target manifestation text and a manifestation text included in each medical examination inference knowledge entry stored in the medical examination inference knowledge base can be specifically calculated, and a specific quantity (that is, Top N, N is the quantity) of medical examination inference knowledge entries with a text similarity reaching a specific threshold or with a highest text similarity can be extracted as the medical examination inference knowledge that matches the target manifestation text.

Alternatively, when the medical examination inference knowledge that matches the target manifestation text is extracted from the medical examination inference knowledge base, feature extraction can be specifically performed on the target manifestation text, to obtain a feature vector corresponding to the target manifestation text, and feature extraction can be performed on a manifestation text included in each medical examination inference knowledge entry stored in the medical examination inference knowledge base, to obtain a feature vector corresponding to the manifestation text included in each medical examination inference knowledge entry. In this case, a vector similarity between the feature vector corresponding to the target manifestation text and the feature vector corresponding to the manifestation text included in each medical examination inference knowledge entry can be calculated, and a specific quantity (that is, Top N, N is the quantity) of medical examination inference knowledge entries with a vector similarity reaching a specific threshold or with a highest vector similarity can be extracted as the medical examination inference knowledge that matches the target manifestation text.

Feature extraction is to extract representative and discriminative information from raw data and convert the information into a form, that is, a feature vector, that can be understood and processed by a machine learning algorithm. When feature extraction is performed on the query text, a word embedding corresponding to the query text can be specifically generated by using a Word2Vec algorithm, as the feature vector corresponding to the query text. Alternatively, the query text can be input into a machine learning model that can be used for text feature extraction, and the machine learning model performs feature extraction on the query text to obtain the feature vector corresponding to the query text. The machine learning model that can be used for text feature extraction can be a convolutional neural network (CNN), or can be a foundation model or a service model of the large language model. This is not specifically limited in this disclosure.

It should be noted that in an offline calculation manner, feature extraction can be performed in advance on the manifestation text included in each medical examination inference knowledge entry stored in the medical examination inference knowledge base, to obtain the feature vector corresponding to the manifestation text included in each medical examination inference knowledge entry, and the obtained feature vector and each medical examination inference knowledge entry can be correspondingly stored. Subsequently, the stored feature vector corresponding to the manifestation text included in each medical examination inference knowledge entry can be directly obtained. Alternatively, in an online calculation manner, after the target manifestation text is obtained, feature extraction can be performed in real time on the manifestation text included in each medical examination inference knowledge entry stored in the medical examination inference knowledge base, to obtain the feature vector corresponding to the manifestation text included in each medical examination inference knowledge entry.

Step 206: Construct a sample based on the extracted medical examination inference knowledge, and construct a prompt text based on the sample and the target manifestation text.

In this embodiment, when the medical examination inference knowledge that matches the target manifestation text is extracted from the medical examination inference knowledge base, a sample can be constructed based on the extracted medical examination inference knowledge, and a limited and small quantity of samples can be constructed. After the sample is constructed, a prompt text of the large language model can be further constructed based on the sample and the target manifestation text.

In some embodiments, the constructed sample can be provided to the model through few-shot learning, that is, before the model receives an actual question, several complete examples including a question and an answer are first displayed, to direct the model to learn how to generate a corresponding answer based on a question. For example, when the prompt text of the large language model is further constructed based on the constructed sample and the target manifestation text, the sample can be placed before the target manifestation text, and context information can be added, so that the large language model can learn from the sample to learn how to generate a corresponding conclusion text based on the manifestation text shown in the sample, and then can perform inference based on the target manifestation text, to generate the corresponding conclusion text.

In some embodiments, as described above, a medical examination inference knowledge entry stored in the medical examination inference knowledge base can include a manifestation text and a conclusion text corresponding to a medical examination, and a descriptive text of an inference step of inferring the conclusion text from the manifestation text. In this case, a limited and small quantity of chain-of-thought samples can be constructed based on the extracted medical examination inference knowledge. After the chain-of-thought sample is constructed, the prompt text of the large language model can be further constructed based on the chain-of-thought sample and the target manifestation text.

A chain of thought is a mode of thinking or problem resolving strategy, is especially applied to the field of artificial intelligence and cognitive science, and emphasizes a step-by-step logical inference process of problem resolving. The chain of thought can be introduced into training and use of the large language model to enhance an interpretation capability and a complex-problem resolving capability of the model by explicitly expressing an intermediate inference step.

The chain of thought emphasizes breaking down a problem resolving process into a series of continuous and logically related steps, including identifying a key element of a problem, listing possible solution paths, and evaluating feasibility of each path until a final answer is reached. Thinking and a reason in each step are included in an input or output of the model, so that a decision process is transparent. This is very critical to understanding decision logic of the model and improving interpretability. An objective of the chain of thought is to make a thinking process of a machine more similar to a human, to resolve a complex problem by imitating how the human thinks step by step, and help the model perform proper inference in the absence of direct training data.

It should be noted that a specific implementation of the chain of thought can involve constructing a training data set that includes an intermediate inference step, or directly embedding such a step-by-step inference format in the prompt of the large language model, to direct the large language model to generate an answer and generate a thinking process or an inference step behind the answer.

The chain-of-thought sample can include key content such as a question statement, an intermediate inference step, and a final answer, and is used to direct and train the large language model to perform step-by-step logical inference and problem resolving.

The question statement is a specific question or task description that needs to be resolved, and can be a mathematical question, logical inference, a fact query, or any query that requires a series of thinking steps to obtain an answer. In the chain-of-thought sample constructed in this embodiment, the question statement can include a manifestation text corresponding to a medical examination.

The intermediate inference step is a core part of the chain-of-thought sample, and includes a series of logical inference, calculation, or analysis for deriving an answer step by step from a question. All steps should be coherent, and a current step should properly lead to a next step until the final answer is reached. For example, resolving a mathematical question may involve formula application, variable replacement, calculation simplification, etc.; and a logical inference task may include precondition analysis, hypothesis verification, etc. In the chain-of-thought sample constructed in this embodiment, the intermediate inference step can include a descriptive text of an inference step of inferring the conclusion text corresponding to the medical examination from the manifestation text corresponding to the medical examination.

The final answer is an answer, to the question, clearly provided after all intermediate inference steps are performed. This answer should be a natural result of an inference chain and be closely connected to the inference steps. In the chain-of-thought sample constructed in this embodiment, the final answer can include the conclusion text corresponding to the medical examination.

In some embodiments, the chain-of-thought sample can direct the model to learn, by using a complete example of a question, an intermediate inference step, and an answer, to imitate such an inference pattern. For example, when the prompt text of the large language model is further constructed based on the constructed chain-of-thought sample and the target manifestation text, the chain-of-thought sample can be placed before the target manifestation text, and context information can be added, so that the large language model first learns from the chain-of-thought sample to learn an inference pattern shown in the chain-of-thought sample, and then imitates such an inference pattern, performs inference based on the target manifestation text, and outputs the inference step in the inference process and the inferred conclusion text.

Step 208: Input the prompt text into the large language model, and output, by using the large language model, a target conclusion text that corresponds to the target medical examination and that is obtained by performing inference based on the target manifestation text and under guidance of the sample.

In this embodiment, when the prompt text is constructed, the prompt text can be input into the large language model, and a conclusion text, that is, the target conclusion text, that corresponds to the target medical examination and that is obtained by performing inference based on the target manifestation text included in the prompt text and under guidance of the sample included in the prompt text can be output by using the large language model. For example, the large language model can first learn from the sample to learn how to generate a corresponding conclusion text based on the manifestation text shown in the sample, and then perform inference based on the target manifestation text, to generate the target conclusion text.

In some embodiments, as described above, the prompt text can be constructed based on the chain-of-thought sample and the target manifestation text. In this case, the large language model can output, in response to the prompt text, a descriptive text (which can be referred to as a target descriptive text) of an inference step of performing inference based on the target manifestation text and under guidance of the chain-of-thought sample, and simultaneously output the inferred target conclusion text. For example, the large language model can first learn from the chain-of-thought sample to learn an inference pattern shown in the chain-of-thought sample, and then imitate such an inference pattern, perform inference based on the target manifestation text, and output the target descriptive text used to describe the inference step in the inference process and the inferred target conclusion text.

In some embodiments, to further improve accuracy of the generated target conclusion text, regular verification can be performed on the descriptive text and/or the target conclusion text output by the large language model.

The regular verification is verification performed based on a regular expression. Regular expression verification is performed on the text generated by the large language model, to ensure that the generated text meets a specific format or pattern requirement, for example, ensure that the generated text meets an expected data format standards, which helps filter out non-compliant content and ensure data quality and consistency. The regular expression is used to match a specific keyword or pattern, to filter out a text that includes a sensitive word or illegal content or that does not conform to policies and regulations, and maintain content compliance. In some highly formatted text generation tasks, the regular expression can be used to check whether there is a grammatical error or an inconsistency in a text.

If the regular verification on the target descriptive text succeeds, the target manifestation text and the target descriptive text can be stored in the medical examination inference knowledge base as medical examination inference knowledge, to update the medical examination inference knowledge stored in the medical examination inference knowledge base.

Alternatively, if the regular verification on the target descriptive text and the target conclusion text succeeds, the target manifestation text, the target conclusion text, and the target descriptive text can be stored in the medical examination inference knowledge base as medical examination inference knowledge, to update the medical examination inference knowledge stored in the medical examination inference knowledge base.

In addition, a medical report corresponding to the target medical examination can be generated based on the target manifestation text and the target conclusion text, and the medical report can be output to a user corresponding to the target medical examination.

In some embodiments, if the regular verification on the target descriptive text and/or the target conclusion text fails, steps 204 to 208 can be performed again. That is, medical examination inference knowledge that matches the target manifestation text can be re-extracted from the medical examination inference knowledge base; a sample (which can be specifically a chain-of-thought sample) can be constructed based on the extracted medical examination inference knowledge, and a prompt text can be constructed based on the sample and the target manifestation text; and the prompt text can be input into the large language model, and a target conclusion text obtained by performing inference based on the target manifestation text and under guidance of the sample can be output by using the large language model; or a target descriptive text of an inference step of performing inference based on the target manifestation text and under guidance of the sample and an inferred target conclusion text can be output by using the large language model.

As described above, a medical examination inference knowledge entry stored in the medical examination inference knowledge base can include a manifestation text and a conclusion text corresponding to a medical examination, and a descriptive text of an inference step of inferring the conclusion text from the manifestation text. The following describes in detail the inference step of inferring the conclusion text from the manifestation text.

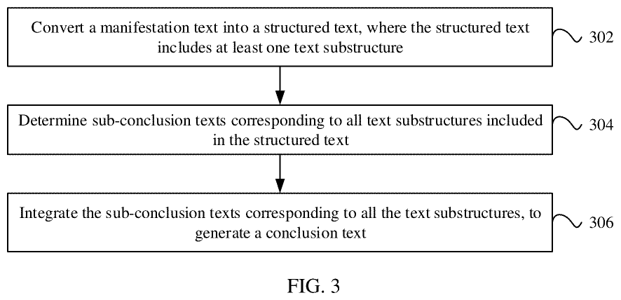

FIG. 3 is a schematic diagram of the inference step of inferring the conclusion text from the manifestation text according to an example embodiment.

As shown in FIG. 3, the inference step of inferring the conclusion text from the manifestation text can include the following steps.

Step 302: Convert the manifestation text into a structured text, where the structured text includes at least one text substructure.

Step 304: Determine sub-conclusion texts corresponding to all text substructures included in the structured text.

Step 306: Integrate the sub-conclusion texts corresponding to all the text substructures, to generate the conclusion text.

In this embodiment, the manifestation text can be first converted into a structured text that includes at least one text substructure; then, sub-conclusion texts corresponding to all text substructures included in the structured text can be determined; and finally, the sub-conclusion texts corresponding to all the text substructures included in the structured text can be integrated, to generate the conclusion text.

In this case, to reduce a data amount of the medical examination inference knowledge stored in the medical examination inference knowledge base and lower storage costs, the descriptive text can be simplified. For example, for a medical examination inference knowledge entry, the medical examination inference knowledge entry can include a manifestation text and a conclusion text corresponding to a medical examination, a structured text into which the manifestation text is converted, and sub-conclusion texts corresponding to all text substructures included in the structured text, as shown in Table 1:

TABLE 1

| | | | |
|---|---|---|---|
| Manifesta- | Text substructure 11 | Sub-conclusion 11 | Conclusion |
| tion text 1 | Text substructure 12 | Sub-conclusion 12 | text 1 |
| | . . . | . . . | |
| | Text substructure 1N | Sub-conclusion 1N | |
| Manifesta- | Text substructure 21 | Sub-conclusion 21 | Conclusion |
| tion text 2 | Text substructure 22 | Sub-conclusion 22 | text 2 |
| | . . . | . . . | |
| | Text substructure 2N | Sub-conclusion 2N | |
| . . . | . . . | . . . | . . . |
| Manifesta- | Text substructure N1 | Sub-conclusion N1 | Conclusion |
| tion text N | Text substructure N2 | Sub-conclusion N2 | text N |
| | . . . | . . . | |
| | Text substructure NN | Sub-conclusion NN | |

Correspondingly, when the chain-of-thought sample is generated based on the simplified medical examination inference knowledge, the context information can be supplemented, so that the structured text into which the manifestation text is converted and the sub-conclusion texts corresponding to all the text substructures included in the structured text are supplemented as the descriptive text of the inference step of inferring the corresponding conclusion text from the manifestation text.

In some embodiments, the text substructure can include a key-value pair. For a text key-value pair, a key in the text key-value pair can be an examined-part identification text, and a value in the text key-value pair can be an examined-part manifestation text.

In some embodiments, when the sub-conclusion texts corresponding to all the text substructures included in the structured text are integrated, to generate the conclusion text, to ensure that the generated conclusion text is simple and clear, sub-conclusion texts indicating an examined-part abnormality in the sub-conclusion texts corresponding to all the text substructures included in the structured text can be concatenated, to generate the conclusion text.

For example, if a manifestation text is “The prostate is increased in size and protrudes upward toward the base of the bladder, no significant abnormal signal is observed in the prostate on T1WI, a nodular iso-to-slightly hyperintense signal is observed in the transition zone on the left side of the prostate on T2WI, DWI shows a high signal, and there is marked heterogeneous enhancement post-contrast administration. The bilateral seminal vesicles show no significant abnormalities, and the vesicoseminal angles are preserved. No significant abnormal signal is observed within the bladder lumen. No markedly enlarged lymph nodes are observed in the pelvic floor and bilateral inguinal regions, with a small amount of fluid in the pelvic cavity. No significant abnormal signal changes are observed in the bony structures of the pelvis. A small abnormal signal is incidentally noted in the left femoral head”, a structured text into which the manifestation text is converted can include the following text key-value pairs:
(prostate, increased in size);
(transition zone on the left side of the prostate, nodular iso-to-slightly hyperintense signal);
(seminal vesicles and vesicoseminal angles, no abnormality);
(bladder lumen, no abnormality);
(pelvic floor and bilateral inguinal regions, no markedly enlarged lymph nodes);
(pelvic cavity, small amount of fluid);
(bony structures of the pelvis, no abnormality); and
(left femoral head, small abnormal signal).

A sub-conclusion text corresponding to the text key-value pair (prostate, increased in size) can be “prostatic hyperplasia”, a sub-conclusion text corresponding to the text key-value pair (transition zone on the left side of the prostate, nodular iso-to-slightly hyperintense signal) can be “PI-RADS classification is considered, prostate cancer is suspected with reference to clinical data, and further evaluation is needed”, a sub-conclusion text corresponding to the text key-value pair (seminal vesicles and vesicoseminal angles, no abnormality) can be “normal”, a sub-conclusion text corresponding to the text key-value pair (bladder lumen, no abnormality) can be “normal”, a sub-conclusion text corresponding to the text key-value pair (pelvic floor and bilateral inguinal regions, no markedly enlarged lymph nodes) can be “normal”, a sub-conclusion text corresponding to the text key-value pair (pelvic cavity, small amount of fluid) can be “There is pelvic effusion that needs to be evaluated with reference to clinical data”, a sub-conclusion text corresponding to the text key-value pair (bony structures of the pelvis, no abnormality) can be “normal”, and a sub-conclusion text corresponding to the text key-value pair (left femoral head, small abnormal signal) can be “It is necessary to consider whether it is related to a tumor, and consideration should be given to further imaging or biomarker testing”. Finally, sub-conclusion texts indicating an examined-part abnormality in these sub-conclusion texts can be concatenated. A generated conclusion text can be “Prostate hyperplasia is noted, PI-RADS classification is considered, prostate cancer is suspected with reference to clinical data, and further evaluation is needed. There is pelvic effusion that needs to be evaluated with reference to clinical data. It is necessary to consider whether it is related to a tumor, and consideration should be given to further imaging or biomarker testing”.

In some embodiments, the generated conclusion text can include not only the sub-conclusion text indicating an examined-part abnormality, but also additional context. The additional context can refer to background knowledge, environment information, context cues, additional descriptions, etc. related to the corresponding manifestation text. The additional context can help the generated conclusion text better follow an intention and meet a requirement, and ensure accuracy and coherence of the conclusion text. In some embodiments, the additional context can be context in the corresponding manifestation text, personal user information, domain knowledge, a spatial-temporal background, implied social and cultural common knowledge, etc.

The above-mentioned example is still used as an example. Finally, the generated conclusion text can be “Prostate hyperplasia is noted. There is a nodular iso-to-slightly hyperintense signal in the transition zone on the left side of the prostate, PI-RADS classification is considered, prostate cancer is suspected with reference to clinical data, and further evaluation is needed. There is a small amount of fluid in the pelvic cavity, which should be evaluated with reference to clinical data. A small abnormal signal is noted in the left femoral head. It is necessary to consider whether it is related to a tumor, and consideration should be given to further imaging or biomarker testing”.

According to the technical solutions provided in the embodiments shown in FIG. 1 to FIG. 3, for a target manifestation text corresponding to a target medical examination, medical examination inference knowledge that matches the target manifestation text can be first extracted from a medical examination inference knowledge base, where the medical examination inference knowledge can include a manifestation text and a conclusion text corresponding to a medical examination; then, a sample can be constructed based on the extracted medical examination inference knowledge, and a prompt text can be further constructed based on the sample and the target manifestation text; and finally, the prompt text can be input into a large language model, and a target conclusion text that corresponds to the target medical examination and that is obtained by performing inference based on the target manifestation text and under guidance of the sample can be output by using the large language model.

In the above-mentioned manner, no doctor or medical professional needs to manually determine a medical examination conclusion corresponding to a medical examination based on a medical examination manifestation corresponding to the medical examination, but the large language model can be used to perform inference based on the medical examination manifestation, to generate a corresponding medical examination conclusion, thereby improving efficiency of generating the medical examination conclusion. In addition, a sample can be generated based on medical examination inference knowledge that matches the medical examination manifestation, and the large language model can learn how to generate a corresponding conclusion text based on a manifestation text shown in the sample, and then perform inference based on the medical examination manifestation, to generate a corresponding medical examination conclusion, thereby improving pertinence of the generated medical examination conclusion and improving accuracy of the generated medical examination conclusion. Moreover, a prompt text of the large language model can be further constructed based on the constructed sample and the medical examination manifestation, that is, the sample can be provided to the large language model through, e.g., few-shot learning, so that the language model can effectively learn on a limited and small quantity of samples, thereby reducing difficulty of training the large language model.

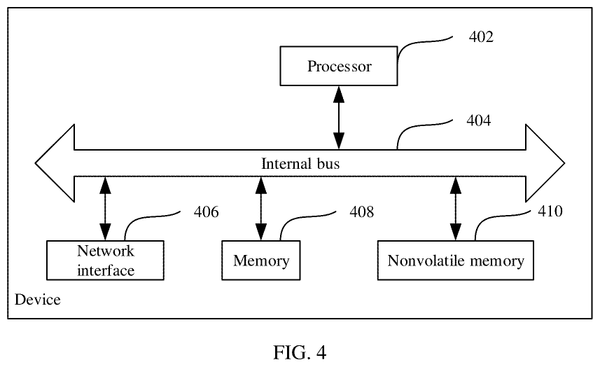

FIG. 4 is a block diagram of an electronic device according to an example embodiment. In terms of hardware, the device includes a processor 402 and a memory 408 storing instructions executable by the processor 402. The device may also include an internal bus 404, a network interface 406, a nonvolatile memory 410, or other hardware as needed. For example, the processor 402 can execute a corresponding computer program stored in the memory 408 to perform the above described method. Accordingly, the device is a large language model-based medical examination conclusion generation apparatus.

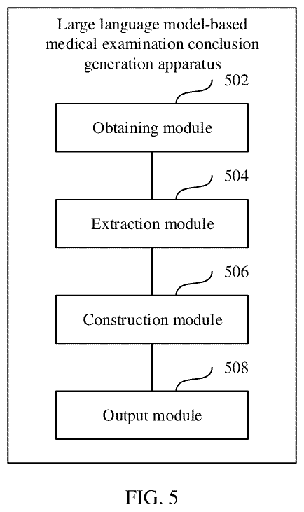

FIG. 5 is a block diagram of a large language model-based medical examination conclusion generation apparatus according to an example embodiment. The apparatus can be applied to the device shown in FIG. 4, and includes:
an obtaining module 502, configured to obtain a target manifestation text corresponding to a target medical examination;
an extraction module 504, configured to extract medical examination inference knowledge that matches the target manifestation text from a medical examination inference knowledge base, where the medical examination inference knowledge includes a manifestation text and a conclusion text corresponding to a medical examination;
a construction module 506, configured to: construct a sample based on the extracted medical examination inference knowledge, and construct a prompt text based on the sample and the target manifestation text; and
an output module 508, configured to: input the prompt text into a large language model, and output, by using the large language model, a target conclusion text that corresponds to the target medical examination and that is obtained by performing inference based on the target manifestation text and under guidance of the sample.

In some embodiments, the medical examination inference knowledge further includes a descriptive text of an inference step of inferring the conclusion text from the manifestation text; and the sample is a chain-of-thought sample; and
the inputting the prompt text into a large language model, and outputting, by using the large language model, a target conclusion text that corresponds to the target medical examination and that is obtained by performing inference based on the target manifestation text and under guidance of the sample includes:
inputting the prompt text into the large language model, and outputting, by using the large language model, a target descriptive text of an inference step of performing inference based on the target manifestation text and under guidance of the chain-of-thought sample and the inferred target conclusion text corresponding to the target medical examination.

In some embodiments, the apparatus further includes:
a verification module, configured to perform regular verification on the target descriptive text and the target conclusion text; and
a storage module, configured to: in response to success of the regular verification, store the target manifestation text, the target conclusion text, and the target descriptive text in the medical examination inference knowledge base as medical examination inference knowledge.

In some embodiments, the apparatus further includes:
a generation module, configured to: in response to success of the regular verification, generate an electronic medical report corresponding to the target medical examination based on the target manifestation text and the target conclusion text, and output the electronic medical report to a user corresponding to the target medical examination.

In some embodiments, the extraction module, the construction module, and the output module are further configured to:
in response to failure of the regular verification, re-extract medical examination inference knowledge that matches the target manifestation text from the medical examination inference knowledge base, construct a chain-of-thought sample based on the extracted medical examination inference knowledge, construct a prompt text based on the chain-of-thought sample and the target manifestation text, and input the prompt text into the large language model.

In some embodiments, the inference step of inferring the conclusion text from the manifestation text includes:
converting the manifestation text into a structured text, where the structured text includes at least one text substructure;
determining sub-conclusion texts corresponding to all text substructures included in the structured text; and
integrating the sub-conclusion texts corresponding to all the text substructures, to generate the conclusion text.

In some embodiments, the text substructure includes a text key-value pair, a key in the text key-value pair is an examined-part identification text, and a value in the text key-value pair is an examined-part manifestation text.

In some embodiments, the integrating the sub-conclusion texts corresponding to all the text substructures, to generate the conclusion text includes:
concatenating sub-conclusion texts indicating an examined-part abnormality in the sub-conclusion texts corresponding to all the text substructures, to generate the conclusion text.

The apparatus embodiments basically correspond to the method embodiments. Therefore, for related parts, references can be made to partial descriptions in the method embodiments. The described apparatus embodiments are merely examples. The modules described as separate parts may or may not be physically separate, and parts displayed as modules may or may not be physical modules, that is, may be located at one position, or may be distributed on a plurality of network modules. Some or all of the modules or units can be selected based on actual requirements to achieve the objectives of the technical solutions of this disclosure.

The system, apparatus, module, or unit described in the above embodiments can be specifically implemented by a computer chip or an entity, or can be implemented by a product having a certain function. An example implementation device is a computer, and a specific form of the computer can be a personal computer, a laptop computer, a cellular phone, a camera phone, a smartphone, a personal digital assistant, a media player, a navigation device, an email receiving/sending device, a game console, a tablet computer, a wearable device, or a combination of any several of these devices. Each module or unit described above can be implemented by hardware, software, or a combination of hardware and software.

In an example configuration, the computer includes one or more central processing units (CPUs), an input/output interface, a network interface, and a memory.

The memory can include a non-persistent memory, a random access memory (RAM), a nonvolatile memory, and/or another form in a computer-readable medium, for example, a read-only memory (ROM) or a flash memory (flash RAM). The memory is an example of the computer-readable medium.

The computer-readable medium includes persistent, non-persistent, removable, and non-removable media that can store information by using any method or technology. The information can be computer-readable instructions, a data structure, a program module, or other data. Examples of the computer storage medium include but are not limited to a phase change random access memory (PRAM), a static random access memory (SRAM), a dynamic random access memory (DRAM), another type of random access memory (RAM), a read-only memory (ROM), an electrically erasable programmable read-only memory (EEPROM), a flash memory or another memory technology, a compact disc read-only memory (CD-ROM), a digital versatile disc (DVD), or another optical storage, a cassette, a disk memory, a quantum memory, a graphene-based storage medium, another magnetic storage device, or any other non-transmission medium. The computer storage medium can be configured to store instructions that, when executed by a processor, cause the processor to perform the above described method. Based on the definition in this specification, the computer-readable medium does not include transitory media such as a modulated data signal and carrier.

It should be noted that the terms “include”, “comprise”, or any other variants thereof are intended to cover a non-exclusive inclusion, so that a method or a device that includes a list of elements not only includes those elements but also includes other elements which are not expressly listed, or further includes elements inherent to such a process, method, product, or device. Without more constraints, an element preceded by “includes a . . . ” does not preclude the presence of additional identical elements in the method or device that includes the element.

Specific embodiments of this disclosure are described above. Other embodiments fall within the scope of this specification. In some cases, actions or steps described in the disclosure can be performed in a sequence different from that in the embodiments and desired results can still be achieved. In addition, the process depicted in the accompanying drawings does not necessarily require a particular sequence or continuous sequence shown to achieve the expected results. In some implementations, multi-tasking and concurrent processing are feasible or may be advantageous.

Terms used in one or more embodiments of this disclosure are merely used to describe specific embodiments, and are not intended to limit the one or more embodiments of this disclosure. The terms “a” and “the” of singular forms are also intended to include plural forms, unless otherwise specified in the context clearly. The term “and/or” indicates and includes any or all possible combinations of one or more associated listed items.

Descriptions of the terms “one embodiment”, “some embodiments”, “example”, “specific example”, or “one implementation” used in one or more embodiments of this disclosure mean that a specific feature or characteristic described with reference to this embodiment is included in at least one embodiment of this disclosure. Schematic descriptions of these terms are not necessarily with respect to a same embodiment. In addition, the described specific feature or characteristic can be combined in a proper manner in one or more embodiments of this disclosure. In addition, without contradicting each other, different embodiments and specific features or characteristics in the different embodiments can be combined.

It should be understood that although terms “first”, “second”, “third”, etc. may be used in one or more embodiments of this disclosure to describe various types of information, the information is not limited to these terms. These terms are merely used to distinguish between information of a same type. For example, without departing from the scope of one or more embodiments of this disclosure, first information can also be referred to as second information, and similarly, the second information can be referred to as the first information. Depending on the context, for example, the word “if” used herein can be explained as “while”, “when”, or “in response to determining”.

The above descriptions are merely example embodiments of this disclosure, but are not intended to limit this disclosure. Any modification, equivalent replacement, improvement, etc. made without departing from the spirit and principle of the one or more embodiments of this disclosure shall fall within the protection scope of the one or more embodiments of this disclosure.

User information (including but not limited to user equipment information, personal user information, etc.) and data (including but not limited to data used for analysis, stored data, displayed data, etc.) in this disclosure are information and data that are authorized by a user or that are fully authorized by each party. Furthermore, related data needs to be collected, used, and processed in compliance with relevant laws, regulations and standards of relevant countries and regions, and corresponding operation entries are provided for the user to choose to authorize or reject.

1. A large language model-based medical examination conclusion generation method, wherein the method comprises:
obtaining a target manifestation text corresponding to a target medical examination;
extracting medical examination inference knowledge that matches the target manifestation text from a medical examination inference knowledge base, wherein the medical examination inference knowledge comprises a manifestation text and a conclusion text corresponding to a medical examination;
constructing a sample based on the extracted medical examination inference knowledge, and constructing a prompt text based on the sample and the target manifestation text; and
inputting the prompt text into a large language model, and outputting, by using the large language model, a target conclusion text that corresponds to the target medical examination and that is obtained by performing inference based on the target manifestation text and under guidance of the sample.

2. The method according to claim 1, wherein the medical examination inference knowledge further comprises a descriptive text of an inference step of inferring the conclusion text from the manifestation text; and the sample is a chain-of-thought sample; and
the inputting the prompt text into a large language model, and outputting, by using the large language model, a target conclusion text that corresponds to the target medical examination and that is obtained by performing inference based on the target manifestation text and under guidance of the sample comprises:
inputting the prompt text into the large language model, and outputting, by using the large language model, a target descriptive text of an inference step of performing inference based on the target manifestation text and under guidance of the chain-of-thought sample and the inferred target conclusion text corresponding to the target medical examination.

3. The method according to claim 2, further comprising:
performing regular verification on the target descriptive text and the target conclusion text; and
in response to success of the regular verification, storing the target manifestation text, the target conclusion text, and the target descriptive text in the medical examination inference knowledge base as medical examination inference knowledge.

4. The method according to claim 3, further comprising:
in response to success of the regular verification, generating an electronic medical report corresponding to the target medical examination based on the target manifestation text and the target conclusion text, and outputting the electronic medical report to a user corresponding to the target medical examination.

5. The method according to claim 3, further comprising:
in response to failure of the regular verification, re-extracting medical examination inference knowledge that matches the target manifestation text from the medical examination inference knowledge base, constructing a chain-of-thought sample based on the extracted medical examination inference knowledge, constructing a prompt text based on the chain-of-thought sample and the target manifestation text, and inputting the prompt text into the large language model.

6. The method according to claim 2, wherein the inference step of inferring the conclusion text from the manifestation text comprises:
converting the manifestation text into a structured text, wherein the structured text comprises at least one text substructure;
determining sub-conclusion texts corresponding to all text substructures comprised in the structured text; and
integrating the sub-conclusion texts corresponding to all the text substructures, to generate the conclusion text.

7. The method according to claim 6, wherein the text substructure comprises a text key-value pair, a key in the text key-value pair is an examined-part identification text, and a value in the text key-value pair is an examined-part manifestation text.

8. The method according to claim 6, wherein the integrating the sub-conclusion texts corresponding to all the text substructures, to generate the conclusion text comprises:
concatenating sub-conclusion texts indicating an examined-part abnormality in the sub-conclusion texts corresponding to all the text substructures, to generate the conclusion text.

9. A large language model-based medical examination conclusion generation apparatus, comprising:
a processor; and
a memory storing instructions executable by the processor,
wherein the processor is configured to:
obtain a target manifestation text corresponding to a target medical examination;
extract medical examination inference knowledge that matches the target manifestation text from a medical examination inference knowledge base, wherein the medical examination inference knowledge comprises a manifestation text and a conclusion text corresponding to a medical examination;
construct a sample based on the extracted medical examination inference knowledge, and construct a prompt text based on the sample and the target manifestation text; and
input the prompt text into a large language model, and output, by using the large language model, a target conclusion text that corresponds to the target medical examination and that is obtained by performing inference based on the target manifestation text and under guidance of the sample.

10. The apparatus according to claim 9, wherein the medical examination inference knowledge further comprises a descriptive text of an inference step of inferring the conclusion text from the manifestation text; and the sample is a chain-of-thought sample; and
the processor is further configured to:
input the prompt text into the large language model, and output, by using the large language model, a target descriptive text of an inference step of performing inference based on the target manifestation text and under guidance of the chain-of-thought sample and the inferred target conclusion text corresponding to the target medical examination.

11. The apparatus according to claim 10, wherein the processor is further configured to:
perform regular verification on the target descriptive text and the target conclusion text; and
in response to success of the regular verification, store the target manifestation text, the target conclusion text, and the target descriptive text in the medical examination inference knowledge base as medical examination inference knowledge.

12. The apparatus according to claim 11, wherein the processor is further configured to:
in response to success of the regular verification, generate an electronic medical report corresponding to the target medical examination based on the target manifestation text and the target conclusion text, and outputting the electronic medical report to a user corresponding to the target medical examination.

13. The apparatus according to claim 11, wherein the processor is further configured to:
in response to failure of the regular verification, re-extract medical examination inference knowledge that matches the target manifestation text from the medical examination inference knowledge base, construct a chain-of-thought sample based on the extracted medical examination inference knowledge, construct a prompt text based on the chain-of-thought sample and the target manifestation text, and input the prompt text into the large language model.

14. The apparatus according to claim 10, wherein the processor is further configured to:
convert the manifestation text into a structured text, wherein the structured text comprises at least one text substructure;
determine sub-conclusion texts corresponding to all text substructures comprised in the structured text; and
integrate the sub-conclusion texts corresponding to all the text substructures, to generate the conclusion text.

15. The apparatus according to claim 14, wherein the text substructure comprises a text key-value pair, a key in the text key-value pair is an examined-part identification text, and a value in the text key-value pair is an examined-part manifestation text.

16. The apparatus according to claim 14, wherein the processor is further configured to:
concatenate sub-conclusion texts indicating an examined-part abnormality in the sub-conclusion texts corresponding to all the text substructures, to generate the conclusion text.

17. A non-transitory computer-readable storage medium storing computer instructions that, when executed by a processor, cause the processor to:
obtain a target manifestation text corresponding to a target medical examination;
extract medical examination inference knowledge that matches the target manifestation text from a medical examination inference knowledge base, wherein the medical examination inference knowledge comprises a manifestation text and a conclusion text corresponding to a medical examination;
construct a sample based on the extracted medical examination inference knowledge, and construct a prompt text based on the sample and the target manifestation text; and
input the prompt text into a large language model, and output, by using the large language model, a target conclusion text that corresponds to the target medical examination and that is obtained by performing inference based on the target manifestation text and under guidance of the sample.
